SEQUENCE LISTING (3 sequences)

SEQ ID NO: 1 - protein, 711 aa, Saccharomyces cerevisiae S288c
MSHFFADHDAPLSMLSVKTEYFPQLTDKEQKYAHFMSKASHAGSRVVMRQVSHESEPIFDLILAIHSKLNGKYPEDDITQKQQTGLYLEYVSQFLSNLGNFKSFGDTKFIPRCEVKFFKQLLELAKINPCSSPLTLSPVDVNHEFTSHHLFSTINELIDIGIYHVEEKAALLGFPSQGYTSAYYLGLPVTPEDMALLKEQLFAELAILPENTRINKVGENSFQIWVASENVKNQITETYPSGQITLSNAVTKVEFIFGDHSREMRLVASYLKEAQKFAANDTQKAMLQEYINHFVTGSSQAHKEAQKLWVKDISPVIETNIGFIETYREPSGIIGEFESLVAIQNKERTAKFSSLVNNAEEFISLLPWSKDYEKPIFNPPDFTSLEVLTFTGSGIPAGINIPNYDDVRLKIGFKNVSLGNILSAAAKSSSKHPPSFISQEDRPIFEKYQSDSFEVQVGIHELLGHGSGKLLTEFTDGFNFDKENPPLGLDGKPVSTYYKVGETWGSKFGQLAGPFEECRAEVIAMFLLTNKKILDIFGFHDVESQDKVIYAGYLQMARAGLLALEYWNPKTGKWGQPHMQARFSIMKTFMKHSTDKNFLKLEMNSTNDDFAIKLDKSLIKTAGHECVKDYLKHLHVYKCSGDVEQGSKYFIDRSTVTPDLASLRDIVLSKRLPRRQFIQSNSYIDDNNKVTLKEYDETPQGMLQSFLDREL

SEQ ID NO: 2 - protein, 711 aa, Artificial Sequence
  Modified sequence from Saccharomyces cerevisiae S288c
MSHFFADHDAPLSMLSVKTEYFPQLTDKEQKYAHFMSKASHAGSRVVMRQVSHESEPIFDLILAIHSKLNGKYPEDDITQKQQTGLYLEYVSQFLSNLGNFKSFGDTKFIPRCEVKFFKQLLELAKINPCSSPLTLSPVDVNHEFTSHHLFSTINELIDIGIYHVEEKAALLGFPSQGYTSAYYLGLPVTPEDMALLKEQLFAELAILPENTRINKVGENSFQIWVASENVKNQITETYPSGQITLSNAVTKVEFIFGDHSREMRLVASYLKEAQKFAANDTQKAMLQEYINHFVTGSSQAHKEAQKLWVKDISPVIETNIGFIETYREPSGIIGEFESLVAIQNKERTAKFSSLVNNAEEFISLLPWSKDYEKPIFNPPDFTSLEVLTFTGSGIPAGINIPNYDDVRLKIGFKNVSLGNILSAAAKSSSKHPPSFISQEDRPIFEKYQSDSFEVQVGIHELLGHGSGKLLTEFTDGFNFDKENPPLGLDGKPVSTYYKVGETWGSKFGQLAGPFEECRAEVIAMFLLTNKKILDIFGFHDVESQDKVIYAGYLQMARAGLLALEYWNPATKKHGQPHMQARFSIMKTFMKHSTDKNFLKLEMNSTNDDFAIKLDKSLIKTAGHECVKDYLKHLHVYKCSGDVEQGSKYFIDRSTVTPDLASLRDIVLSKRLPRRQFIQSNSYIDDNNKVTLKEYDETPQGMLQSFLDREL

SEQ ID NO: 3 - DNA, 2100 bp, Artificial Sequence
  Synthesized
atgagccactttttcgccgatcatgatgctcctctgagcatgctttctgttaaaacagaatactttcctcaattgactgataaggaacaaaaatatgcgcatttcatgtcaaaggcctcccatgcgggttcaagggttgtaatgagacaagtttctcatgagagtgagccaatttttgacctaatccttgccattcattcaaagctaaacggcaagtacccagaggacgatattacgcagaagcagcaaacgggtttgtatttggaatacgtttctcaattcttatctaatttgggtaatttcaaatcgtttggtgacactaagtttattcctcgttgtgaggtaaaattcttcaaacagcttttggagctggccaagattaatccgtgttcttctccgctcactttatctcctgttgacgttaaccatgaattcacatctcatcatcttttttccaccatcaatgagctaattgatattggtatttaccatgtggaagagaaggcggctctcttagggtttccctctcaaggttatacttcagcctattatctgggtttacctgtgacacctgaagatatggctcttttgaaagagcagttgtttgctgaacttgccatcttgcctgaaaacacaagaatcaacaaagttggtgaaaacagtttccaaatctgggttgcctctgagaatgtgaaaaaccagataacagaaacctaccccagtggacagatcacattatccaatgctgtaaccaaagtagaattcatttttggtgatcattcacgtgaaatgcgtttagtagcatcgtatttaaaggaagctcaaaaattcgcggctaatgatactcaaaaagcaatgcttcaggaatacatcaaccactttgtcactggctcttctcaagcacataaagaagcacaaaaactttgggtcaaagatatatctcccgtcattgaaacaaatatcggttttatcgaaacatatagagaaccctcgggcataattggagaatttgaatcgttggttgcaattcaaaacaaagaacgtactgctaaattttccagcttggttaacaacgcagaagaattcatttccttactaccatggtctaaagattacgaaaaaccgattttcaatccaccagatttcacctctctagaagtattaacgtttactggatcgggtataccagcgggcatcaatattccaaactatgatgatgttcggcttaaaattgggttcaagaatgtttctttggggaatatcttaagcgcggctgccaaaagctcatccaagcatccgccaagttttatatcgcaagaagatcgcccaatttttgaaaaatatcaaagtgattcttttgaagtccaagtaggcatccatgaattattaggacatggttcaggaaagttgttgacagaatttacagacggctttaattttgataaggaaaaccctcctttaggtttggatgggaaaccggtgagcacatactacaaagttggtgaaacttggggttccaaatttggacagttagctggcccatttgaagaatgtcgtgcggaagtaattgccatgtttttgcttactaataagaagattcttgatatttttggtttccatgatgtcgaatctcaagataaagtgatctacgctggatatctacaaatggcccgtgcgggtctcctagctttagaatactggaatccagctactaagaagcaaggacaaccacacatgcaagcaagattttctatcatgaaaacatttatgaagcactctacagataagaatttcttaaagttggagatgaacagcacgaatgatgattttgccatcaagttggataaatctctcattaaaacagcgggacatgaatgtgtgaaagactatttaaagcatttgcatgtttacaaatgttcaggcgatgtggaacagggaagtaagtactttattgatagatcaacggtgacaccggatttggcgtctttaagagacatcgtcttatctaagagattgccaaggagacaattcatacaatcgaattcttatattgacgacaataacaaggtaaccctgaaagaatatgatgaaaccccacag

(19) United States
(12) Patent Application Publication
(10) Pub. No.: US 2019/0246664 A1
(43) Pub. Date: Aug. 15, 2019

(54) SITE-SPECIFIC MUTAGENESIS MODIFIED YEAST DIPEPTIDYL PEPTIDASE III

(71) Applicant: GUANGDONG GENUIZYMES ANIMAL HEALTH CO., LTD, Kaiping, Jiangmen, Guangdong (CN)

(72) Inventors: Daling LIU, Guangzhou, Guangdong (CN); Dongsheng YAO, Guangzhou, Guangdong (CN); Xiyang WU, Guangzhou, Guangdong (CN); Chunfang XIE, Guangzhou, Guangdong (CN)

(73) Assignee: GUANGDONG GENUIZYMES ANIMAL HEALTH CO., LTD, Kaiping, Jiangmen, Guangdong (CN)

(21) Appl. No.: 15/780,903

(22) PCT Filed: Nov. 28, 2016
(86) PCT No.: PCT/CN2016/107454

§ 371 Date: Jun. 1, 2018

(30) Foreign Application Priority Data
Dec. 9, 2015 (CN) 201510908511.8

(51) Int. Cl. A23K 20/189; C12N 9/48; A61K 38/48; C12R 1/85

(52) U.S. Cl. CPC A23K 20/189; C12N 9/485; C12Y 304/14004; C12R 1/85; A61K 38/48

(57) ABSTRACT

The invention relates to a site-specific mutagenesis modified yeast dipeptidyl peptidase III. The yeast dipeptidyl peptidase III is an isolated mutant produced by making a plurality of amino acid substitutions in a wild type yeast dipeptidyl peptidase III derived from Saccharomyces cerevisiae S288c having an amino acid sequence of SEQ ID NO. 1, said amino acid substitutions comprising substitutions at positions 570, 572 and 574, so that the site-specific mutagenesis modified yeast dipeptidyl peptidase III is an enzyme having an oxidative decomposition activity on 6-methoxy-bifuran coumarin. The site-specific mutagenesis modified yeast dipeptidyl peptidase III can be used in preparation of a feed and additives thereof, and a food and additives thereof, in which 6-methoxy difuran coumarin is eliminated, and can be used in preparation of a medicament for preventing diseases induced by 6-methoxy-difuran coumarin.

### FIELD OF INVENTION

The invention relates to a dipeptidyl peptidase III, in particular to a site-specific mutagenesis modified yeast dipeptidyl peptidase III.

### BACKGROUND OF INVENTION

The dipeptidyl peptidase III (DPPs III, EC 3.4.14.4), such as a yeast dipeptidyl peptidase III, a human dipeptidyl peptidase III, and a mouse dipeptidyl peptidase III from, a rabbit dipeptide enzyme III and the like, is a group of metalloprotease containing a special HEXXGH zinc finger structure in its molecule, and is a peptide enzyme having a hydrolyzed polypeptide chain with a cut-down dipeptide amino tail end. The DPP III relates to the physiological function of the metabolism of enkephalin and the angiotensin II, angiotensin III, melanin and the other important physiological active peptides. The DPPS is present in tissues of various mammals, and is divided into different types according to the positioning of subcells, the particular sensitivity of a nuclear inhibitor. The DPP III can selectively hydrolyze the dipeptide residues from the N-terminal of the polypeptide chain or protein, such as Arg-Arg-, Ala-Arg- or Tyr-Gry. The DPP III derived from the yeast is composed of 712 amino acids, and the zinc ions are catalytic metal ions. There is 37% homology in the amino acid sequence of between DPP III and aflatoxin monooxygenase (AFMO), but the DPP III does not have the function of oxidizing and decomposing 6-methoxy-bifuran coumarin.

Aflatoxin is mainly a high-toxicity secondary metabolite generated by fungi such as Aspergillus flavus and Aspergillus parasitic. There are eight kinds of structures of the Aflatoxin molecules mainly determined, wherein Aflatoxin B1 (also known as 6-methoxy-bifuran coumarin) has the strongest toxicity, it is considered that Aflatoxin B1 is a kind of strong carcinogenic mutagenic agent which is extremely prominent in harm to human beings. A large amount of intake of Aflatoxin B1 to people or animal will cause acute poisoning reaction, and even death. A small-dose long-term intake can lead to teratogenesis, mutation and carcinogenicity. Only dozens of ppb level of Aflatoxin B1 still has great toxicity. Family I Aflatoxin contains a furan double-bond structure. At present, it has been found that seldom biological enzymes have a decomposition activity on 6-methoxy-bifuran coumarin. Aflatoxin monooxygenase (AFMO) is an enzyme which has an oxidative decomposition activity on 6-methoxy-bifuran coumarin. Researches show that the process of oxidizing and decomposing the 6-methoxy-bifuran coumarin by the AFMO is as following: transmitting electrons from the substrate molecules to oxygen, reducing the water into hydrogen peroxide, oxidizing the substrate and further opening the furan double-bond in the molecule. During the process, electron transfer is realized by valence change of valence ions in the molecule, and the method comprises the following steps: firstly, the two-valence metal ions on AFMO are combined to capture one electron of the substrate, and the substrate itself is changed into a monovalent ion, and then the unstable monovalent ions transmit the obtained electrons to oxygen, and the unstable monovalent ions are transformed into stable bivalent ions, the oxygen molecule is used for obtaining hydrogen peroxide under the participation of water molecules, and meanwhile, the substrate is converted into the epoxide of the hydrogen peroxide. Then, the epoxide is subjected to oxidative hydrolysis reaction with the action of hydrogen peroxide, and finally, the furan double bonds in the substrate molecule are disconnected. The aflatoxin monooxygenase is a currently reported biological enzyme for detoxification of aflatoxin. Therefore, it is very important in the development of the aflatoxin reduction technology to find and produce a novel enzyme with the oxidative decomposition activity on 6-methoxy-bifuran coumarin.

### SUMMARY OF THE INVENTION

It is a primary object to provide a site-specific mutagenesis modified yeast dipeptidyl peptidase III, and the yeast dipeptidyl peptidase III mutant has an oxidative decomposition function on 6-methoxy-bifuran coumarin.

According to a first aspect, there is provided a site-specific mutagenesis modified yeast dipeptidyl peptidase III, wherein the yeast dipeptidyl peptidase III is an isolated mutant produced by making a plurality of amino acid substitutions in a wild type yeast dipeptidyl peptidase III derived from Saccharomyces cerevisiae S288c having an amino acid sequence of SEQ ID NO. 1 (NCBI Database ID No. NM_001183312), said amino acid substitutions comprising substitutions at positions 570, 572 and 574, so that the site-specific mutagenesis modified yeast dipeptidyl peptidase III is an enzyme having an oxidative decomposition activity on 6-methoxy-bifuran coumarin.

According to the further technical feature of the site-specific mutagenesis modified yeast dipeptidyl peptidase III of the present invention, the amino acid substitution at position 570 is a Alanine (Ala, A) amino acid residue for a Lysine (Lys, K) amino acid residue, the amino acid substitution at position 572 is a Lysine (Lys, K) amino acid residue for a Glycine (Gly, G) amino acid residue, and the amino acid substitution at position 574 is a Histidine (His, H) amino acid residue for a Tryptophan (Trp, W) amino acid residue, said site-specific mutagenesis modified yeast dipeptidyl peptidase III having an amino acid sequence of SEQ ID NO. 2.

Under the experimental verification, the site-specific mutagenesis modified yeast dipeptidyl peptidase III (hereinafter referred to as “myDPP”) has an oxidative decomposition activity on 6-methoxy-bifuran coumarin. Under the condition of pH6.0, the reaction temperature of 25° C., after 50 minutes, the oxidative decomposition efficiency of the mutant enzyme on 6-methoxy-bifuran coumarin (100 ppb) is up to 90%. The other enzymatic properties of the mutant enzyme are similar to that of wild type enzyme.

According to a second aspect, there is provided an isolated DNA molecule encoding a site-specific mutagenesis modified yeast dipeptidyl peptidase III of the present invention.

Preferably, the DNA molecule of the present invention comprises a nucleotide sequence of SEQ ID NO. 3.

According to a third aspect, there is provided a recombinant expression vector comprising the DNA molecule of the present invention.

According to a fourth aspect, there is provided a host cell comprising the DNA molecule of the present invention, or the recombinant expression vector of the present invention. The recombinant expression vector and the transformed host cell can be prepared by the well-known technical means in the art.

According to a fifth aspect, there is provided a method for producing a site-specific mutagenesis modified yeast dipeptidyl peptidase III according to claim 1, said method comprising: cultivating the transformed host cell according to claim 6 under conditions suitable for expression of the dipeptidyl peptidase III; and separating, purifying and recovering the mutant yeast dipeptidyl peptidase III.

The DNA molecule of the present invention can be inserted into the vector or the expression system in proper orientation and correct reading frame, and then is transferred into the host cell, the DNA molecule can be expressed in any eukaryotic or prokaryotic expression system. A variety of host-vector systems may be utilized to express the protein-encoding sequence(s). Preferred host-vector systems include but are not limited to the following: bacteria transformed with phage, vector or cosmid; microorganisms such as yeast containing yeast vectors; mammalian cell systems infected with virus; insect cell systems infected with virus; and plant cells infected by bacteria. Preferred vectors include a viral vector, plasmid, cosmid or an oligonucleotide.

The preferable host is a eukaryotic system, such as pichia pastoris. The preferred protein expression method comprises the following steps: inducing secretory expression of pichia pastoris by methanol.

The inventors successfully obtain a site-specific mutagenesis modified yeast dipeptidyl peptidase III, and under the activity identification experiment, it is shown that the dipeptidyl peptidase III mutant has an oxidative decomposition activity on 6-methoxy-bifuran coumarin which does not shown in the wild type dipeptidyl peptidase III, and the biological activity is enough to be applied in preparation of an animal feed as well as an additive, a food, or a medicament.

According to a sixth aspect, there is provided a use of the site-specific mutagenesis modified yeast dipeptidyl peptidase III according to claim 1 in preparation of a feed and additives thereof, and a food and additives thereof, in which 6-methoxy difuran coumarin is eliminated. Based on the existing processing technology of feed and food, the site-specific mutagenesis modified yeast dipeptidyl peptidase III can be added into the animal feed for feed detoxification as a detoxication agent, or be made into a immobilized enzyme for removing toxicity of foods such as peanut oil, or be producing probiotics or probiotic microcapsule which can express the enzyme, and be used for removing toxicity of food, grain and oil, feed and the like.

According to a seventh aspect, there is provided use of the site-specific mutagenesis modified yeast dipeptidyl peptidase III according to claim 1 in preparation of a medicament for preventing diseases induced by 6-methoxy-difuran coumarin. Based on the conventional medicine preparation process, the site-specific mutagenesis modified yeast dipeptidyl peptidase III of the invention can be used for preventing the yeast dipeptidyl peptidase III which is used for preventing 6-methoxy-bifuran coumarin induced diseases (such as tumors).

### BRIEF DESCRIPTION OF THE DRAWINGS

FIG. 1 is an identification diagram of a recombinant vector myDPP expression plasmid.

FIG. 2 is a schematic drawing of purification result of the recombinant myDPP and the recombinant wtyDPP.

### DETAIL DESCRIPTION OF THE INVENTION

The terms used in the present disclosure, unless otherwise indicated, are the meanings commonly understood by those skilled in the art. Definitions of some special terms used in the invention are provided in the following.

The “wtyDPP” refers to a wild type yeast dipeptidyl peptidase III, and its gene is represented by an italicized wtyDPP.

The “myDPP” refers to a mutant yeast dipeptidyl peptidase III, and its gene is represented by an italicized myDPP.

### Example 1: Synthetise of wtyDPP and myDPP

According to the invention, based on the gene sequence (NCBI Database, NM_001183312) of dipeptidyl peptidase from Saccharomyces cerevisiae S288C, 5′-GTCGAATTC-3′ is added at the 5′ end, and 3′-CCTAGGGAC-5′ is added at the 3′ end, “GAATTC” is a restriction site of EcoRI, and “GGATCC” is a restriction site of BamHI.

The gene wtyDPP is synthesized by an artificial all-synthesis method.

According to the invention, based on the gene sequence (NCBI Database, NM_001183312) of dipeptidyl peptidase from Saccharomyces cerevisiae S288C, the amino acid residue at position 570 is replaced by Alanine (Ala, A) amino acid residue, and the amino acid residue at position 572 is replaced by Lysine (Lys, K) amino acid residue, and the amino acid residue at position 574 is replaced by Histidine (His, H) amino acid residue. 5′-GTCGAATTC-3′ is added at the 5′ end, and 3′-CCTAGGGAC-5′ is added at the 3′ end, “GAATTC” is a restriction site of EcoRI, and “GGATCC” is a restriction site of BamHI. The gene myDPP is synthesized by an artificial all-synthesis method.

After the site-specific mutagenesis modification, Alanine (Ala, A) at position 570, Lysine (Lys, K) at position 572, Histidine (His, H) at position 574, and Glutamine (Gln, Q) at position 576, Histidine (His, H) at position 578, Methionine (Met, M) at position 579, Glutamine (Gln, Q) at position 580, Alanine (Ala, A) at position 581 and Arginine (Arg, R) at position 582 forms a sequence AXKXHXQXHMQAR, wherein A is Alanine, R is Arginine, X is any amino acid.

The gene synthesis is completed by a commercial company, for example, Shanghai Jierui Biological Co., Ltd.

### Example 2: Construction of Recombinant Expression Vector for wtyDPP and myDPP

Gene cloning was carried out according to a conventional method (Sambrook, et al. 2001, molecular cloning a laboratory manual. Cold spring harbor laboratory press. USA). Genes wtyDPP and myDPP obtained from Example 1 were respectively cloned to an expression vector pHIL-S1 to build two recombinant expression vectors pHIL-S1-wtyDPP and pHIL-S1-myDPP. The cloned target genes were identified by restriction enzymatic incisions and sequencing.

Detailed Steps of the Method

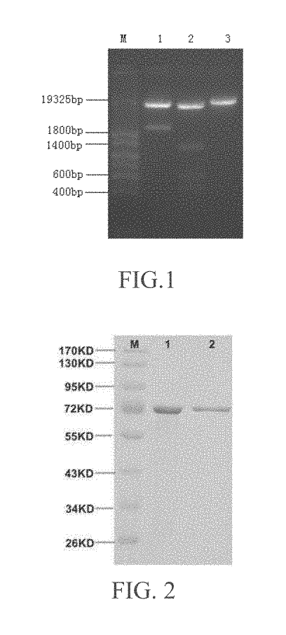

The construction procedure of the recombinant vector pHIL-S1 containing myDPP was as following: EcoRI+BamHI double enzymatic incisions were made on the vector pHIL-S1 and the target segment myDPP, and the enzymatic incision product was isolated by 0.8% agarose gel electrophoresis, and then the product was cut from the gel and recovered. Let the vector pHIL-S1 connect with the myDPP by T4 DNA ligase. E. coli DH5a competent cells were prepared using CaCl₂ method, and then were transformed by the recombinant vectors. The transformants were screened, and the recombinant vectors were extracted from the screened transformants. The recombinant vector pHIL-S1-myDPP was identified by EcoRI+BamHI, HindIII, SacI restriction enzymatic incisions. The vector DNA was extracted and purified form the recombinant vector by PEG purification method (Sambrook, et al 2001, Molecular Cloning A Laboratory Manual. Cold Spring Harbor Labroratory Press. USA). T7 and SP6 were used as sequencing primers, and the DNA was sequenced in forward and reverse directions by using DNA automatic sequencer. The result of the restriction enzyme digestion of the recombinant vector pHIL-S1-myDPP was shown in FIG. 1, the EcoRI+BamHI double restriction enzymatic incisions product (Sample 1) was a band about 2100 bp in the gel, and HindIII single restriction enzymatic incision product (Sample 2) and SacI single restriction enzymatic incision product (Sample 3) were shown in the figure.

The construction procedure of the recombinant vector pHIL-S1 containing wtyDPP was as following: the target segment myDPP was replaced by wtyDPP during the construction procedure of the recombinant vector pHIL-S1 containing myDPP, and the other operations is the same as that in the construction procedure of the recombinant vector pHIL-S1 containing myDPP.

### Example 3: Expression of Recombinant myDPP and Recombinant wtyDPP

The expression of the recombinant myDPP was as follows: SacI restriction enzymatic incision was made on the recombinant vector pHIL-S1-myDPP and the vector pHIL-S1, the enzymatic incision product was isolated by 0.8% agarose gel electrophoresis, and then the product was cut from the gel. The linear recombinant vector pHIL-S1-myDPP and the vector pHIL-S1 were recovered. The pichia pastoris GS115 was transformed by a spheroplast method according to the handbook of Pichia Expression Kit (Invitrogen Inc., USA), and the Mut⁺ transformants were screened. The recombinant bacteria were induced to expression by using methanol as a unique carbon source, according to the operations of the handbook of Pichia Expression Kit. The result of SDS-PAGE electrophoresis shows that there was obvious target protein band appeared in the supernatant of the culture after the induced expression. And there was no target protein band appeared in the supernatant of the culture of the negative control bacteria containing empty vector after 96 hours under the same condition. The results were shown in FIG. 2, Sample 1 was myDPP, and Sample 2 was wtyDPP. After induced by methanol, obvious target protein band was appeared on the supernatant of the culture. However, there was not obvious target protein band appeared in the supernatant of the culture of the negative control bacteria containing empty vector after the induced expression under the same condition.

Detailed Steps of the Method

The homologous recombination of the recombinant and pichia pastoris

I. Linearization of the Vector

The recombinant vector pHIL-S1-myDPP and the vector pHIL-S1 were cleaved with SacI restriction enzyme. The linearized vector pHIL-S1 will be used as a control of the following experiments.

The enzymatic incision of pHIL-S1-myDPP (120 μl total system): 12 μl of Buffer L+8 μl SacI+100 μl/pHIL-S1-myDPP.

The enzymatic incision of pHIL-S1 (120 μl total system): 12 μl of Buffer L+8 μl SacI+100 μl pHIL-S1.

Samples were recovered by a 0.8% agarose gel electrophoresis, and then cut from the gel, and the recombinant vector pHIL-S1-myDPP and the vector pHIL-S1 were recovered.

II. Culture of Pichia Pastoris GS115 Used for Spheroplast Culture

The method comprises the following steps:

1. a GS115 monoclone was selected from a flat plate and was inoculated into 10 ml YPD (Yeast Extract Peptone Dextrose medium). Culturing overnight at 30° C. in a shaking 150 ml conical incubator (250-300 rpm).

2. respectively taking 5, 10, 20 μl bacteria liquid from 10 ml overnight YPD culture and then inoculating into 200 ml YPD. Culturing overnight in a shaking 500 ml conical incubator (250-300 rpm).

3. The three cultures were detected for OD600. The ones with OD600=0.2-0.3 were selected, and pelleted by centrifugation at 1500×g for 5 minutes at room temperature. The supernatant was discarded. The collected cells were used for spheroplast transformation.

III. Preparation of Pichia pastoris GS115 Spheroplasts

1. The cell pellet was re-suspended in 200 ml sterile water, and then transferred to two 10 ml sterile centrifuge tubes.

2. The cells were pelleted by centrifugation at 1500×g for 5 min at room temperature. The supernatant was discarded.

3. The cell pellet was washed with fresh prepared SED, followed by centrifugation at 1500×g for 5 min at room temperature. The supernatant was discarded.

4. The cell pellet was washed with 1M Sorbitol solution, followed by centrifugation at 1500×g for 5 min at room temperature. The supernatant was discarded.

5. The cell pellet was re-suspended in 10 ml SCE.

6. Zymolyase in a tube was thawed and mixed by flicking the tube.

7. 7.5 μl of Zymolyase was added into the cells and the cells were incubated for 30 min at 30° C.

8. The cells were pelleted by centrifugation at 750×g for 5 min at room temperature. The supernatant was discarded.

9. The transformation mixture was washed with 1M Sorbitol solution, mixed by flicking the tube to disperse the precipitate. The cells were pelleted by centrifugation at 750×g for 10 min at room temperature. The supernatant was discarded, and the cell pellet was collected.

10. The cell pellet was washed with 10 ml CaS solution, followed by centrifugation at 750×g for 5 min. The supernatant was discarded.

11. The cell pellet was re-suspended in 0.6 ml CaS solution. The spheroplasts must be used within 30 min.

IV. Spheroplast Transformation of Pichia pastoris GS115

1. Aliquots of 100 μl each of Pichia pastoris GS115 spheroplasts were respectively transferred to three 15 ml sterile centrifuge tubes A, B and C.

2. Tube A (no DNA) as negative control, tube B (added 30 μl linearized vector pHIL-S1), tube C (added 30 μl linearized recombinant plasmid pSA, incubated for 10 min at room temperature). 3 ml of fresh PEG/CaT was prepared at the same time.

3. Aliquots of 1 ml each of fresh PEG/CaT were added to tubes A, B and C, mixed gently and incubated for 10 min at room temperature.

4. The cells were pelleted by centrifugation at 750×g for 5 min at room temperature. The supernatant was discarded.

5. The cell pellets were re-suspended in 150 μl SOS, incubated for 20 min at room temperature.

6. Aliquots of 850 μl 1 M Sorbitol solution each were added to the tubes. 7. The entire transformations were plated on RD solid incubation plates using a sterile spreader (200 μl/plate). The plates were incubated at 28-30° C. Transformants were appeared between 4-6 days.

V. Selection of Mut⁺ Transformants

1. Using a sterile toothpick, His+ transformants were patched on both MM and MD plates, the strains GS115/His⁺Mut^{s} Albumin and GS115/His⁺Mut⁺β-gal were also patched on the plates as controls.

2. Plates were incubated at 28-30° C. for 2 days.

3. After two days, scored both MM and MD plates. Mut⁺ strains will grow normally on both MM and MD plates, while Mut^{s} will grow normally only on the MD plate but little or no growth on MM plate.

VI. Induced Expression of the Recombinant Strains

1. Inoculated a single colony of His⁺Mut⁺ transformant in 25 ml BMG in a 250 ml baffled flask. Grew at 28-30° C. in a shaking incubator (250-300 rpm) until the culture reached OD600=2-6 (˜16-18 h).

2. Cells were harvested by centrifugation at 1500-3000×g for 5 min at room temperature. Supernatant was decanted and cell pellet was re-suspended in BMM to an OD600 of 1.0 (˜100-200 ml BMM). The culture was placed in a 1-litter baffled flask and returned to incubator to continue growth at 250-300 rpm at 28-30° C.

3. 100% methanol was added to a final concentration of 0.5% to maintain inducted expression.

4. After 96 h, the expression culture was centrifuged for 2-3 min, supernatant was transferred to a separate tube and stored at −80° C. for purification of expression product.

The supernatant of the culture after 96 h induction was analyzed. Total mount of protein was 0.23 mg/ml. The molecular weight of the protein product is consistent with the predicted value of 78 kDa by BioEdit.

The expression of the recombinant wtyDPP is as follows: the recombinant vector in the expression process of the recombinant myDPP is replaced by the vector pHIL-S1-wtyDPP, and the other operations are the same as that of the recombinant myDPP.

### Example 4: Purification of Recombinant myDPP and wtyDPP

The recombinant expression culture was precipitated with 70% saturation (NH₄)₂SO₄, producing crude enzyme as precipitate. The crude enzyme was dissolved in equal volume of PBS, centrifuged. The supernatant was loaded on a hydrophobic Phenyl Sepharose column; active products were collected from gradient elution. The product was subjected to dialysis desalination and concentrated after equilibration with PBS. The active peak was eluted using pH gradient and fraction collected. The PBS solution is concentrated after being balanced. The method specifically comprises the following steps:

I. Crude Enzyme from (NH₄)₂SO₄ Precipitation

(NH₄)₂SO₄ powder was added to the recombinant expression culture until 40% saturation followed by centrifugation at 10000 g for 20 min at 4° C. The supernatant was added more (NH₄)₂SO₄ until 70% saturation. Crude enzyme was obtained from centrifugation at 10000 g for 20 min at 4° C.

II. Hydrophobic Interaction Chromatography

The crude enzyme was dissolved in equal volume of 0.02 M PBS (pH 6.0). and centrifuged at 4000 g for 10 min at 4° C. Supernatant was loaded on a Phenyl Sepharose column (Pharmacia Biotech. Inc.) which had been washed to background using 0.02M PBS+30% saturation (NH₄)₂SO₄, pH 6.0. Gradient elution with solution A (0.02M PBS+10% saturation (NH₄)₂SO₄, pH 6.0) and solution B (0.02 M PBS, pH 6.0) gave an active product. The product was subjected to dialysis desalination and concentrated after equilibration with F solution (0.02 M PBS+5 M NaCl, pH 7.5) to 1 mg/ml. The object product peak is identified by SDS-PAGE electrophoresis.

### Example 5: Test of the Oxidative Decomposition Activity of Recombinant Proteins myDPP and wtyDPP on 6-methoxy-bifuran coumarin

The enzyme unit (U) is a unit for the amount of a particular enzyme. One U is defined as the amount of the enzyme that produces 1 μmol H₂O₂ that is, the amount that catalyzes the conversion of 1 micro mole of substrate per minute.

The method of measuring the enzyme activity:

30 μl of substrate (6-methoxy-bifuran coumarin) with the concentration of 100 μg/ml into 10 ml of enzyme with the concentration of 10 μg/ml. Let the mixture react for 10 minutes at 25° C. and pH6.5, and then 200 μl of Horseradish Peroxidase (HRP) with the concentration of 0.34 mg/ml and 200 μl of 3, 3′-5, 5′-tetramethyl benzidine (TMB) with the concentration of 5 mM were added, and then developing for 30 minutes, then measuring the light absorption value at the ultraviolet of 650 nm, and calculating the enzyme activity unit.

The enzyme activity result shows that protein wtyDPP has no decomposition activity on 6-methoxy-bifuran coumarin, but the protein myDPP has a decomposition activity on 6-methoxy-bifuran coumarin, and its relative enzyme activity is 33.61 U/mg.

TABLE 1
Sample Processing Method

| | Reaction System | |
|---|---|---|
| | Group | |
| Steps of the Method | wtyDPP | Group myDPP |
| 1. wtypDPP enzyme solution was added. | 10 ul | — |
| 2. myDPP enzyme solution was added. | — | 10 ul |
| 3. 6-methoxy-bifuran coumarin solution was | 30 ul | 30 ul |
| added. | | |

4. The reaction was carried out at the pH 6.5 and 25° C. for 30 minutes.

| | | |
|---|---|---|
| 5. Horseradish peroxidase was added. | 200 ul | 200 ul |
| 6. 3,3′-5,5′-tetramethyl benzidine was added. | 200 ul | 200 ul |
| 7. Methyl alcohol was added. | 200 ul | 200 ul |

8. The mixture was subjected to color while mixing for 30 minutes, and
then the light absorbance value was measured in 650 nm.

1. A site-specific mutagenesis modified yeast dipeptidyl peptidase III, wherein the yeast dipeptidyl peptidase III is an isolated mutant produced by making a plurality of amino acid substitutions in a wild type yeast dipeptidyl peptidase III derived from Saccharomyces cerevisiae S288c having an amino acid sequence of SEQ ID NO. 1, said amino acid substitutions comprising substitutions at positions 570, 572 and 574, so that the site-specific mutagenesis modified yeast dipeptidyl peptidase III is an enzyme having an oxidative decomposition activity on 6-methoxy-bifuran coumarin.

2. The site-specific mutagenesis modified yeast dipeptidyl peptidase III according to claim 1, wherein the amino acid substitution at position 570 is a Alanine (Ala, A) amino acid residue for a Lysine (Lys, K) amino acid residue, the amino acid substitution at position 572 is a Lysine (Lys, K) amino acid residue for a Glycine (Gly, G) amino acid residue, and the amino acid substitution at position 574 is a Histidine (His, H) amino acid residue for a Tryptophan (Trp, W) amino acid residue, said site-specific mutagenesis modified yeast dipeptidyl peptidase III having an amino acid sequence of SEQ ID NO. 2.

3. An isolated DNA molecule encoding a site-specific mutagenesis modified yeast dipeptidyl peptidase III according to claim 2.

4. The isolated DNA molecule according to claim 3, wherein the DNA molecule comprises a nucleotide sequence of SEQ ID NO. 3.

5. A recombinant expression vector comprising a DNA molecule according to claim 3.

6. A host cell comprising a DNA molecule according to claim 3.

7. A method for producing a site-specific mutagenesis modified yeast dipeptidyl peptidase III according to claim 1, said method comprising: cultivating the transformed host cell according to claim 6 under conditions suitable for expression of the dipeptidyl peptidase III; and separating, purifying and recovering the mutant yeast dipeptidyl peptidase III.

8. A use of the site-specific mutagenesis modified yeast dipeptidyl peptidase III according to claim 1 in preparation of a feed and additives thereof, and a food and additives thereof, in which 6-methoxy difuran coumarin is eliminated.

9. A use of the site-specific mutagenesis modified yeast dipeptidyl peptidase III according to claim 1 in preparation of a medicament for preventing diseases induced by 6-methoxy-difuran coumarin.

10. A recombinant expression vector comprising a DNA molecule according to claim 4.

11. A host cell comprising a DNA molecule according to claim 4.

12. A host cell comprising a recombinant expression vector according to the claim 11.
